(19) United States
(12) Patent Application Publication
(10) Pub. No.: US 2024/0377383 A1
(43) Pub. Date: Nov. 14, 2024

(54) ANALYTE MONITORING SYSTEM AND METHODS OF USE

(71) Applicant: ABBOTT DIABETES CARE INC., Alameda, CA (US)

(72) Inventors: Wesley Scott Harper, Alameda, CA (US); Erwin Satrya Budiman, Fremont, CA (US); Kenneth J. Doniger, Menlo Park, CA (US); Gary Alan Hayter, Oakland, CA (US)

(73) Assignee: ABBOTT DIABETES CARE INC., Alameda, CA (US)

(21) Appl. No.: 18/784,287

(22) Filed: Jul. 25, 2024

Related U.S. Application Data
(63) Continuation of application No. 17/868,386, filed on Jul. 19, 2022.
Application No. 17/868,386 is a continuation of application No. 16/111,099, filed on Aug. 23, 2018, now Pat. No. 11,391,723.
Application No. 16/111,099 is a continuation of application No. 15/143,413, filed on Apr. 29, 2016, now Pat. No. 10,082,493.
Application No. 15/143,413 is a continuation of application No. 13/683,996, filed on Nov. 21, 2012, now Pat. No. 9,339,217.
(60) Provisional application No. 61/563,743, filed on Nov. 25, 2011.

(51) Int. Cl. G01N 33/487; A61B 5/00; A61B 5/145; A61M 5/172; G01N 33/49; G16Z 99/00

(52) U.S. Cl. CPC G01N 33/48792; A61B 5/0004; A61B 5/0026; A61B 5/14503; A61B 5/1451; A61B 5/14532; A61B 5/4839; A61B 5/6833; A61B 5/7225; A61B 5/7275; A61M 5/1723; G01N 33/49; G16Z 99/00; A61B 5/002

(57) ABSTRACT

The present disclosure provides methods of processing data provided by a transcutaneous or subcutaneous glucose sensor utilizing different algorithms to strike a balance between signal responsiveness accompanied by signal noise and the introduction of error associated with that noise. The methods utilize the strengths of a lag correction algorithm and a smoothing algorithm to optimize the quality and value of the resulting data (glucose concentrations and the rates of change in glucose concentrations) to a continuous glucose monitoring system. Also provided are systems and kits.

### RELATED APPLICATION

The present application is a continuation of U.S. patent application Ser. No. 17/868,386 filed Jul. 19, 2022, which is a continuation of U.S. patent application Ser. No. 16/111,099 filed Aug. 23, 2018, now U.S. Pat. No. 11,391,723, which is a continuation of U.S. patent application Ser. No. 15/143,413 filed Apr. 29, 2016, now U.S. Pat. No. 10,082,493, which is a continuation of U.S. patent application Ser. No. 13/683,996 filed Nov. 21, 2012, now U.S. Pat. No. 9,339,217, which claims priority to U.S. Provisional Application No. 61/563,743, filed Nov. 25, 2011, entitled “Analyte Monitoring System and Methods of Use”, the disclosures of each of which are incorporated by reference for all purposes.

### BACKGROUND

Analyte, e.g., glucose monitoring systems including continuous and discrete monitoring systems, generally include a small, lightweight, battery powered and microprocessor controlled system configured to detect signals proportional to the corresponding measured glucose levels using an electrometer and radio frequency (RF) signals to transmit the collected data. Certain analyte monitoring systems include a transcutaneous or subcutaneous analyte sensor configuration which is, for example, partially mounted on the skin of a subject whose analyte level is to be monitored. The sensor cell may use a two or three-electrode (work, reference, and counter electrodes) configuration driven by a controlled potential (potentiostat) analog circuit connected through a contact system.

The analyte sensor may be configured so that a portion thereof is placed under the skin of the patient so as to detect the analyte levels of the patient, with another segment of the analyte sensor adapted to be in communication with the data processing unit. The data processing unit is configured to communicate the analyte levels detected by the sensor over a wireless communication link such as an RF (radio frequency) communication link to a receiver/monitor unit. The receiver/monitor unit performs data analysis, among others on the received analyte levels to generate information pertaining to the monitored analyte levels.

For systems that calculate the blood glucose concentration by measuring interstitial glucose in real time, lag between the interstitial glucose and blood glucose and sensor noise can introduce error, some of which is detrimental to obtaining accurate glucose and glucose rate of change data.

It would be desirable to have a method and system to account for lag and system noise and provide robust and useful data that is relevant to the blood glucose system.

### SUMMARY

The present disclosure describes embodiments including methods for overcoming sensor noise and lag between the interstitial fluid and blood, both of which can introduce error. Certain embodiments allow for a balance between signal responsiveness and the reduction in noise by a smoothing process. Certain aspects utilize a combination of algorithms, including algorithms optimized to maintain minimal expected error between the final sensor output and a corresponding reference and one that provides more stable rates of change in glucose levels. In certain aspects, lag correction algorithms designed to minimize the correlation between sensor error (with respect to reference blood glucose) and rates of change calculate glucose values with optimal point wise accuracy, and smoothing algorithms to minimize the effect of noise accurately calculate rates of change in glucose levels.

The present disclosure provides embodiments including methods for monitoring an analyte including monitoring a data stream including a set of contiguous source data points related to the analyte concentration; if a higher sample rate is desired, generating a set of monitored data streams from the set of contiguous source data points, utilizing one or more of the monitored data streams or the set of contiguous source data points to compute a maximum lag correction oriented signal, wherein the lag correction algorithm attempts to eliminate the expected error due to blood-to-interstitial glucose dynamics with reasonable but minimal regard to noise amplification, utilizing one or more of the monitored data streams or the set of contiguous source data points to generate a maximum smoothing oriented signal, wherein the smoothing algorithm attempts to eliminate noise, which includes sample time-to-sample time variation not correlated to true glucose change, utilizing the maximum lag correction oriented signal for generating analyte concentration and utilizing the maximum smoothing oriented signal for generating a rate of change in the analyte concentration.

Certain embodiments of the present disclosure include methods for monitoring an analyte including monitoring a data stream including a set of contiguous source data points related to the analyte concentration, providing a set of monitored data streams from the set of source data points, wherein each set of monitored data streams is generated utilizing a spline, up-sampling, or regularization algorithm, providing a set of maximum lag corrected signals from the set of monitored data streams, wherein each set of maximum lag corrected signals is generated utilizing correction terms based on time derivative estimates and historical monitored data stream data points that minimize the correlation between the expected glucose error and time derivative estimates or minimize the correlation between the expected glucose error and a predetermined array of historical monitored data stream, providing a set of maximum smoothing signals from the set of monitored data streams, wherein each set of maximum smoothing signals is generated utilizing a smoothing algorithm, generating analyte concentration utilizing the maximum lag corrected signals, and generating a rate of change in the analyte concentration utilizing the maximum smoothing signals. In certain aspects, rate of change can be computed based on maximum smoothing signals using a variety of methods, including a backwards first difference approximation using the most recent two successive maximum smoothing signals measurements, Finite Impulse Response (FIR) filters employing 2 or more recent maximum smoothing signals measurements, or the slope of a Least Squares Error Fit of a line based on recent maximum smoothing signals measurements.

Certain aspects of the present disclosure include methods for monitoring an analyte including monitoring a data stream including a set of contiguous source data points related to analyte concentration, providing a set of maximum lag corrected signals from the source data points, wherein each set of maximum lag corrected signals is generated utilizing correction terms based on time derivative estimates and historical source data points that minimize the correlation between the expected glucose error and time derivative estimates or minimize the correlation between the expected glucose error and a pre-determined array of historical source data points, providing a set of maximum smoothing signals from the set of source data points, wherein each set of maximum smoothing signals is generated utilizing a smoothing algorithm, generating analyte concentration utilizing the maximum lag corrected signals, and generating a rate of change in the analyte concentration utilizing the maximum smoothing signals.

Aspects of the present disclosure include methods for monitoring an analyte including monitoring a data stream including a set of contiguous source data points related to the analyte concentration, providing a set of maximum lag corrected signals from the set of monitored data stream, wherein each set of maximum lag corrected signals is generated utilizing correction terms based on time derivative estimates and historical monitored data stream data points that minimize the correlation between the expected glucose error and time derivative estimates or minimize the correlation between the expected glucose error and a pre-determined array of historical monitored data stream, providing a set of maximum smoothing signals from the set of monitored data stream, wherein each set of maximum smoothing signals is generated utilizing a smoothing algorithm, providing a second set of maximum smoothing signals from the set of monitored data stream, wherein each of the second set of maximum smoothing signals is generated utilizing a smoothing algorithm, generating analyte concentration utilizing a weighted combination of maximum lag corrected signals and maximum smoothing signals, wherein more weight is placed on the set of maximum lag corrected signals to generate the output, and generating a rate of change in the analyte concentration utilizing a weighted combination of maximum lag corrected signals and maximum smoothing signals, where more weight is placed on the maximum smoothing signals to generate the output.

Certain aspects of the present disclosure include methods for monitoring an analyte comprising monitoring a data stream including a set of contiguous source data points related to the analyte concentration, providing a set of maximum lag corrected signals from the set of source data points, wherein each set of maximum lag corrected signals is generated utilizing correction terms based on time derivative estimates and historical source data points that minimize the correlation between the expected glucose error and time derivative estimates or minimize the correlation between the expected glucose error and a pre-determined array of historical source data points, providing a set of maximum smoothing signals from the set of source data points, wherein each set of maximum smoothing signals is generated utilizing a smoothing algorithm, generating analyte concentration utilizing a weighted combination of maximum lag corrected signals and maximum smoothing signals, where more weight is placed on the maximum lag corrected signals to generate the output and generating a rate of change in the analyte concentration utilizing a weighted combination of maximum lag corrected signals and maximum smoothing signals, where more weight is placed on the maximum smoothing signals to generate the output.

### BRIEF DESCRIPTION OF THE DRAWINGS

FIG. 1 shows a data monitoring and management system such as, for example, an analyte (e.g., glucose) monitoring system in accordance with certain embodiments of the present disclosure;

FIG. 2 illustrates data related to changes in interstitial fluid measurement and two filtered outputs as compared against discrete blood glucose measurements and an up-sampled or monitored smooth blood glucose signal;

FIG. 3 illustrates a closer view of a region from FIG. 2, highlighting data during a rapid rise in glucose level illustrating how each algorithm deals with a rapid rise in glucose level;

FIG. 4 is a flow chart illustrating certain embodiments for monitoring and processing analyte data;

FIG. 5 is a flow chart illustrating certain embodiments for monitoring and processing analyte data utilizing monitored data stream; and

FIG. 6 is a flow chart illustrating certain embodiments for monitoring and processing analyte data utilizing weighted data processing.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, exemplary methods and materials are now described. All publications mentioned herein are incorporated herein by reference to disclose and describe the methods and/or materials in connection with which the publications are cited.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

The present disclosure provides for the utilization of a combination of algorithms to strike a balance between responsiveness and minimizing noise. Certain embodiments involve a maximum lag correction algorithm and a maximum smoothing algorithm. The lag correcting algorithm minimizes the expected point-wise error of glucose values, resulting in accurate glucose values whereas the smoothing algorithm minimizes the sample time-to-sample time variability which provides rates of change with optimum accuracy. The development and use of lag correction algorithms can be found in U.S. Patent Publication Nos. 2010/0191085, now U.S. Pat. No. 8,224,415, 2010/0023291, now U.S. Pat. No. 8,515,517, 2009/0198118, now U.S. Pat. Nos. 8,473,022, and 7,618,369, the disclosures of each of which are incorporated herein by reference for all purposes.

In certain embodiments, algorithms compensate for lag between blood glucose (BG) and interstitial glucose (IG), measured by a sensor, by correlating time derivatives (e.g. rate estimate, acceleration estimate, band limited rate estimate, etc.) with the BG to IG discrepancy, correlating a pre-determined array of historical sensor data with the BG to IG discrepancy, and providing cancellation terms computed from the sensor signal. These algorithms can improve aggregate point wise accuracy of a continuous glucose monitoring system relative to blood glucose values. However, a consequence of the improved responsiveness is an increased susceptibility to noise due to the amplification of errors caused by noise in the time derivative calculations that are necessary to produce a rate estimate. To better estimate the rate of change, algorithms can be developed that smooth recent data to remove artifacts from noise.

In certain embodiments, lag correction algorithms selected vary in aggressiveness in order to strike a balance between levels of responsiveness and smoothing. A lag correction algorithm is deemed more aggressive when it attempts to remove the average error correlated to blood-to-interstitial glucose dynamics with less regard to the noise amplification effect it generates. A lag correction algorithm is overly aggressive if the amount of lag correction exceeds the required amount, resulting in an error of the opposite sign as compared to when no lag correction is attempted. A lag correction algorithm is deemed less aggressive if the amount of correction is somewhere between the optimal amount and no lag correction at all.

The combination of a lag correcting algorithm in combination with a smoothing algorithm can similarly be used to provide more accurate acceleration data in combination with representative glucose values.

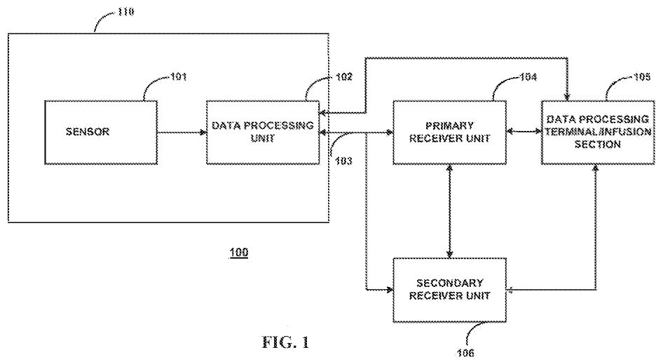

FIG. 1 shows a data monitoring and management system such as, for example, an analyte (e.g., glucose) monitoring system in accordance with certain embodiments of the present disclosure. Embodiments of the subject disclosure are described primarily with respect to glucose monitoring devices and systems, and methods of using two or more devices in a glucose monitoring system to determine the compatibility of one or more devices in the glucose monitoring system.

Analytes that may be monitored include, but are not limited to, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times.

Referring to FIG. 1, the analyte monitoring system 100 includes a sensor 101, a data processing unit (e.g., sensor electronics) 102 connectable to the sensor 101, and a primary receiver unit 104 which is configured to communicate with the data processing unit 102 via a communication link 103. In aspects of the present disclosure, the sensor 101 and the data processing unit (sensor electronics) 102 may be configured as a single integrated assembly 110. In certain embodiments, the integrated sensor and sensor electronics assembly 110 may be configured as an on-body patch device. In such embodiments, the on-body patch device may be configured for, for example, RFID or RF communication with a reader device/receiver unit, and/or an insulin pump.

In certain embodiments, the primary receiver unit 104 may be further configured to transmit data to a data processing terminal 105 to evaluate or otherwise process or format data received by the primary receiver unit 104. The data processing terminal 105 may be configured to receive data directly from the data processing unit 102 via a communication link which may optionally be configured for bi-directional communication. Further, the data processing unit 102 may include a data processing unit or a transceiver to transmit and/or receive data to and/or from the primary receiver unit 104, the data processing terminal 105 or optionally the secondary receiver unit 106.

Also shown in FIG. 1 is an optional secondary receiver unit 106 which is operatively coupled to the communication link and configured to receive data transmitted from the data processing unit 102. The secondary receiver unit 106 may be configured to communicate with the primary receiver unit 104, as well as the data processing terminal 105. The secondary receiver unit 106 may be configured for bidirectional wireless communication with each of the primary receiver unit 104 and the data processing terminal 105. As discussed in further detail below, in certain embodiments the secondary receiver unit 106 may be a de-featured receiver as compared to the primary receiver unit 104, i.e., the secondary receiver unit 106 may include a limited or minimal number of functions and features as compared with the primary receiver unit 104. As such, the secondary receiver unit 106 may include a smaller (in one or more, including all, dimensions), compact housing or embodied in a device such as a wrist watch, arm band, etc., for example. Alternatively, the secondary receiver unit 106 may be configured with the same or substantially similar functions and features as the primary receiver unit 104. The secondary receiver unit 106 may include a docking portion to be mated with a docking cradle unit for placement by, e.g., the bedside for night time monitoring, and/or bi-directional communication device.

Only one sensor 101, data processing unit 102 and data processing terminal 105 are shown in the embodiment of the analyte monitoring system 100 illustrated in FIG. 1. However, it will be appreciated by one of ordinary skill in the art that the analyte monitoring system 100 may include more than one sensor 101 and/or more than one data processing unit 102, and/or more than one data processing terminal 105.

The analyte monitoring system 100 may be a continuous monitoring system, or semi-continuous, or a discrete monitoring system. In a multi-component environment, each component may be configured to be uniquely identified by one or more of the other components in the system so that communication conflict may be readily resolved between the various components within the analyte monitoring system 100. For example, unique IDs, communication channels, and the like, may be used.

In certain embodiments, the sensor 101 is physically positioned in or on the body of a user whose analyte level is being monitored. The sensor 101 may be configured to at least process send data related to its configuration into a corresponding signal for transmission by the data processing unit 102.

The data processing unit 102 is coupleable to the sensor 101 so that both devices are positioned in or on the user's body, with at least a portion of the analyte sensor 101 positioned transcutaneously. The data processing unit 102 in certain embodiments may include a portion of the sensor 101 (proximal section of the sensor in electrical communication with the data processing unit 102) which is encapsulated within or on the printed circuit board of the data processing unit 102 with, for example, potting material or other protective material. The data processing unit 102 performs data processing functions, where such functions may include but are not limited to, filtering and encoding of data signals, each of which corresponds to a sampled analyte level of the user, for transmission to the primary receiver unit 104 via the communication link 103. In one embodiment, the sensor 101 or the data processing unit 102 or a combined sensor/data processing unit may be wholly implantable under the skin layer of the user.

In one aspect, the primary receiver unit 104 may include an analog interface section including an RF receiver and an antenna that is configured to communicate with the data processing unit 102 via the communication link 103, and a data processing section for processing the received data from the data processing unit 102 such as data decoding, error detection and correction, data clock generation, and/or data bit recovery.

In operation, the primary receiver unit 104 in certain embodiments is configured to synchronize with the data processing unit 102 to uniquely identify the data processing unit 102, based on, for example, an identification information of the data processing unit 102, and thereafter, to periodically receive signals transmitted from the data processing unit 102 associated with the monitored analyte levels detected by the sensor 101. That is, when operating in the CGM mode, the receiver unit 104 in certain embodiments is configured to automatically receive data related to the configuration of the sensor from the analyte sensor/sensor electronics when the communication link (e.g., RF range) is maintained or opened between these components.

Referring again to FIG. 1, the data processing terminal 105 may include a personal computer, portable data processing devices or computers such as a laptop computer or a handheld device (e.g., personal digital assistants (PDAs), communication devices such as a cellular phone (e.g., a multimedia and Internet-enabled mobile phone such as an iPhone, a Blackberry device, a Palm device such as Palm Pre, Treo, or similar phone), mp3 player, pager, and the like), drug delivery device, insulin pump, each of which may be configured for data communication with the receiver via a wired or a wireless connection. Additionally, the data processing terminal 105 may further be connected to a data network (not shown).

The data processing terminal 105 may include an infusion device such as an insulin infusion pump or the like, which may be configured to administer insulin to patients, and which may be configured to communicate with the primary receiver unit 104 for receiving, among others, the measured analyte level or configuration data. Alternatively, the primary receiver unit 104 may be configured to integrate an infusion device therein so that the primary receiver unit 104 is configured to administer insulin (or other appropriate drug) therapy to patients, for example, for administering and modifying basal profiles, as well as for determining appropriate boluses for administration based on, among others, the detected analyte levels received from the data processing unit 102. An infusion device may be an external device or an internal device (wholly implantable in a user).

In particular embodiments, the data processing terminal 105, which may include an insulin pump, may be configured to receive the configuration signals from the data processing unit 102, and thus, incorporate the functions of the primary receiver unit 104 including data processing for managing the patient's insulin therapy and analyte monitoring. In certain embodiments, the communication link 103 as well as one or more of the other communication interfaces shown in FIG. 1 may use one or more of an RF communication protocol, an infrared communication protocol, a Bluetooth® enabled communication protocol, an 802.11x wireless communication protocol, or an equivalent wireless communication protocol which would allow secure, wireless communication of several units (for example, per HIPAA requirements) while avoiding potential data collision and interference.

The analyte monitoring system may include an on-body patch device with a thin profile that can be worn on the arm or other locations on the body (and under clothing worn by the user or the patient), the on-body patch device including an analyte sensor and circuitry and components for operating the sensor and processing and storing signals, including configuration signals, received from the sensor as well as for communication with the reader device. For example, one aspect of the on-body patch device may include electronics to sample the voltage signal received from the analyte sensor in fluid contact with the body fluid, and to process the sampled voltage signals into the corresponding glucose values and/or store the sampled voltage signal as raw data, or to send configuration information as a signal or data.

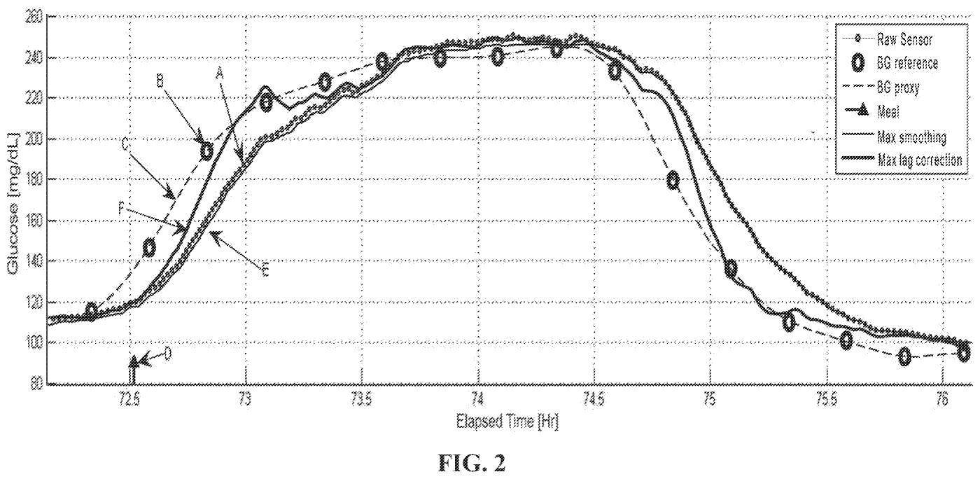

FIG. 2 illustrates data related to changes in blood glucose levels and related data generated from an interstitial fluid measurement, such as interstitial fluid measurements taken by the analyte monitoring system of FIG. 1, processed utilizing an aggressive lag correction algorithm and a smoothing algorithm. Lag from blood glucose (BG) to interstitial glucose (IG) is illustrated in FIG. 2. The raw sensor signal measures glucose concentration in the interstitial fluid space. BG reference collected at a slower sample rate is up-sampled as BG proxy to visually match the raw sensor's sample time. Both the BG and BG proxy data are only used to provide context on some of the characteristics of IG compared to BG. Neither the BG nor BG proxy data are needed for the methods described in this invention. IG levels match BG well provided that glucose concentrations remain steady. Otherwise, IG levels are generally lower than BG while glucose increases, and higher while glucose decreases. From a point-wise perspective, this reduced responsiveness manifests itself as one source of sensor error. However, any attempt to correct for this lag results in the amplification of noise, as can be seen in the maximum lag correction line (curve F in FIG. 2). Even in the absence of lag correction, it may be necessary to process the sensor signal to filter out noise. A smoothed IG signal is shown to reduce the sample time-to-sample time fluctuations, but further increases the overall BG to IG lag, which is especially visible in curve E in FIG. 2 while glucose is increasing. The term smoothing implies the reduction of sample time-to-sample time fluctuations, but does not require the process to be limited to a retrospective operation.

Referring to FIG. 2, the first curve A represents raw sensor data. BG measurements B are up-sampled into a smooth curve C in order to visually demonstrate a more fluid progression of BG data. A meal event D is shown to increase glucose. The fifth curve E represents data processed with a smoothing curve. The sixth curve F provides output that is close to the blood glucose levels represented by curve C. Raw sensor signal is collected at a pre-determined time interval (e.g. once every 1 minute) where the signal is either: (a) pre filtered at a much faster time interval and/or processed to remove erroneous data or (b) a direct sampling from the source without any preprocessing. Blood glucose is also collected at a longer time interval (e.g. once every 15 minutes) to provide reference data. A smooth BG proxy is generated from the BG reference at a sample interval and time points that match the pre-determined time interval of the collected sensor signal. Both the BG and BG proxy data are only used to provide context, and are not needed for the methods described in this invention. A meal event initiates a rise in the patient's BG level. Maximizing the signal noise filtering is shown to produce a smoother signal at the expense of additional lag. On the other hand, maximizing lag correction allows for better point-wise agreement with BG at the expense of additional sample-to-sample variation.

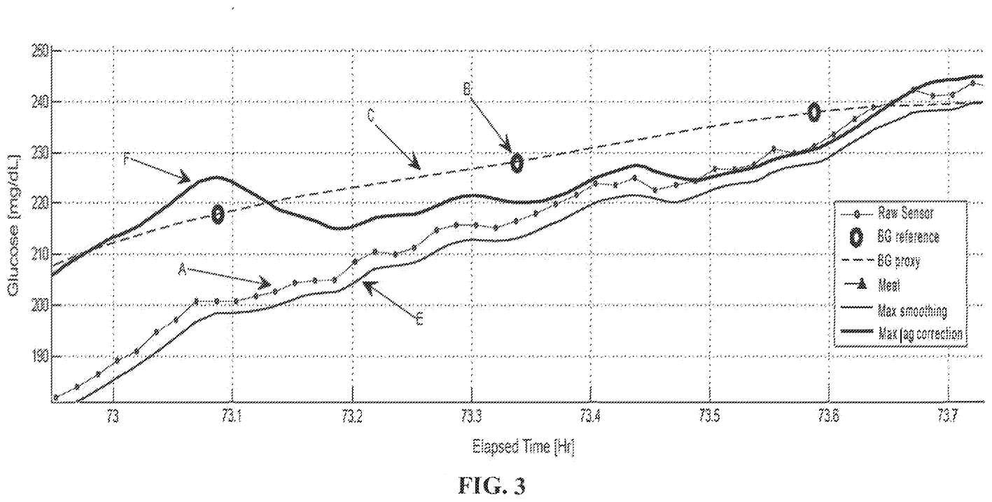

FIG. 3 illustrates a closer view of a region (73.0 to 73.7 units of elapsed time) from FIG. 2, highlighting data during a rapid rise in glucose level illustrating how each algorithm deals with a rapid rise in glucose level. The highlighted region of FIG. 2, provided in FIG. 3, focuses on a region of rapid increase in glucose and illustrates the different way each algorithm responds. For example, during the rapid increase in glucose, curve E, generated without any lag correction, provides more accurate rate of change data because its trend matches well with that of the trend of curve C, but provides less accurate data regarding glucose concentration. In contrast, curve F generated with lag correction produces data output regarding glucose levels during the rapid increase in glucose levels that closely corresponds to curve C, representing actual blood glucose data.

Aspects of the present disclosure provide for the utilization of the combination of a lag correction algorithm and a smoothing algorithm in a continuous glucose monitoring system to optimize the accuracy of readings related to glucose levels and the rates of change in glucose levels. The achievement of better accuracy for readings of both glucose level and its rate of change is important in first understanding the real changes in blood glucose and properly managing its level.

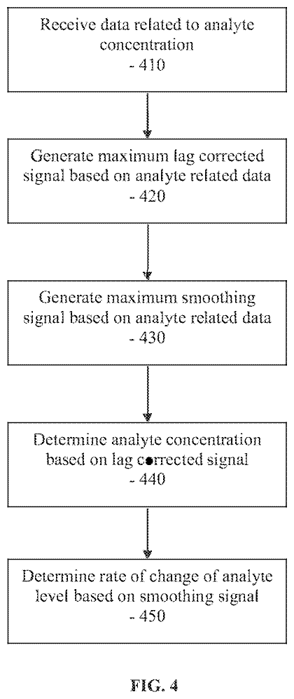

FIG. 4 is a flow chart illustrating certain embodiments for monitoring and processing analyte data. Referring to FIG. 4, a processing unit, such as data processing unit 102 or receiver unit 104 of the analyte monitoring system 100 of FIG. 1, receives data related to an analyte concentration (410), which may be received from a sensor, such as sensor 101 of analyte monitoring system 100 of FIG. 1. The received data is analyzed and a maximum lag corrected signal is generated (420). In certain embodiments, the maximum lag corrected signal is generated by utilizing correction terms based on time derivative estimates and historical source data points, and wherein parameters for the maximum lag correction minimize the correlation between the expected glucose error and time derivative estimates and minimize the correlation between the expected glucose error and a pre-determined array of historical source data points. In certain embodiments, the maximum lag correction signal is generated by utilizing an aggressive lag correction algorithm configured to drive one or more of the correlation between the expected glucose error and the time derivative estimates or the correlation between the expected glucose error and a predetermined array of historical data points, to zero. The maximum lag corrected signal is analyzed to determine a corrected analyte concentration (440).

Referring still to FIG. 4, the received data is also analyzed and a maximum smoothing signal is generated (430) by applying a smoothing algorithm to the received data and the maximum smoothing signal is analyzed to determine the rate of change of the monitored analyte level (450).

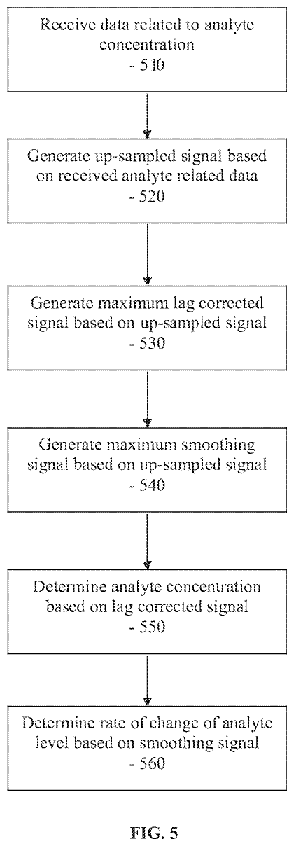

FIG. 5 is a flow chart illustrating certain embodiments for monitoring and processing analyte data utilizing monitored data stream. Referring to FIG. 5, a processing unit, such as data processing unit 102 or receiver unit 104 of the analyte monitoring system 100 of FIG. 1, receives data related to an analyte concentration (510), which may be received from a sensor, such as sensor 101 of analyte monitoring system 100 of FIG. 1. The received data is sampled at a high speed to generate a monitored data stream (520). In certain embodiments, the monitored data stream is optionally generated utilizing a spline, up-sampling, or regularization algorithm. The monitored data stream is analyzed and a maximum lag corrected signal is generated (530). In certain embodiments, the maximum lag corrected signal is generated by utilizing correction terms based on time derivative estimates and historical source data points, and wherein parameters for the maximum lag correction minimize the correlation between the expected glucose error and time derivative estimates and minimize the correlation between the expected glucose error and a pre-determined array of historical source data points. In certain embodiments, the maximum lag correction signal is generated by utilizing an aggressive lag correction algorithm configured to drive one or more of the correlation between the expected glucose error and the time derivative estimates or the correlation between the expected glucose error and a pre-determined array of historical data points, to zero. The maximum lag corrected signal is analyzed to determine a corrected analyte concentration (550).

Referring still to FIG. 5, the received data is also analyzed and a maximum smoothing signal is generated (540) by applying a smoothing algorithm to the received data and the maximum smoothing signal is analyzed to determine the rate of change of the monitored analyte level (560).

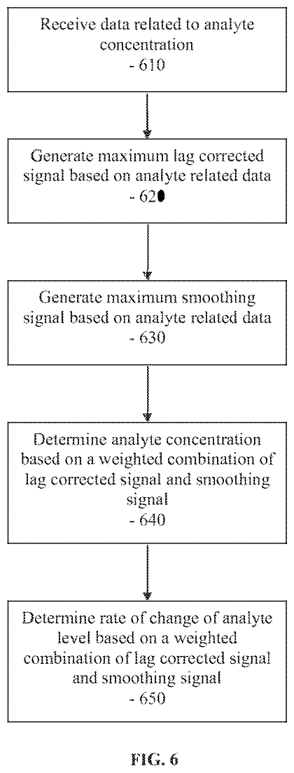

FIG. 6 is a flow chart illustrating certain embodiments for monitoring and processing analyte data utilizing weighted data processing. Referring to FIG. 6, a processing unit, such as data processing unit 102 or receiver unit 104 of the analyte monitoring system 100 of FIG. 1, receives data related to an analyte concentration (610), which may be received from a sensor, such as sensor 101 of analyte monitoring system 100 of FIG. 1. The received data is analyzed and a maximum lag corrected signal is generated (620). In certain embodiments, the maximum lag corrected signal is generated by utilizing correction terms based on time derivative estimates and historical source data points, and wherein parameters for the maximum lag correction minimize the correlation between the expected glucose error and time derivative estimates and minimize the correlation between the expected glucose error and a predetermined array of historical source data points. In certain embodiments, the maximum lag correction signal is generated by utilizing an aggressive lag correction algorithm configured to drive one or more of the correlation between the expected glucose error and the time derivative estimates or the correlation between the expected glucose error and a pre-determined array of historical data points, to zero.

Referring still to FIG. 6, the received data is also analyzed and a maximum smoothing signal is generated (630) by applying a smoothing algorithm to the received data. After determination of the maximum lag corrected signal and the maximum smoothing signal, both signals are analyzed and weighted to determine the analyte concentration (640) and rate of change of the monitored analyte level (650). In certain embodiments, the analyte concentration determination is made by weighting the maximum lag corrected signal more heavily than the maximum smoothing signal and the rate of change determination is made by weighting the maximum smoothing signal more heavily than the maximum lag corrected signal.

In certain embodiments, there is provided a method for monitoring an analyte comprising monitoring a data stream including a set of contiguous source data points related to the concentration of an analyte, providing one or more sets of maximum lag corrected signals from the one or more sets of monitored data stream, wherein each set of maximum lag corrected signals is generated utilizing correction terms based on time derivative estimates and historical monitored data stream data points, and wherein parameters for the maximum lag correction minimize the correlation between the expected glucose error and time derivative estimates and minimize the correlation between the expected glucose error and a pre-determined array of historical monitored data stream, providing one or more sets of maximum smoothing signals from the set of monitored data stream, wherein each set of maximum smoothing signals is generated utilizing a smoothing algorithm, determining analyte concentration utilizing the one or more sets of maximum lag corrected signals, and determining a rate of change in the analyte concentration utilizing the one or more sets of maximum smoothing signals.

In certain aspects, providing the one or more sets of maximum lag corrected signals includes utilizing an aggressive lag correction algorithm, the lag correction algorithm configured to drive one or more of the correlation between the expected glucose error and the time derivative estimates or the correlation between the expected glucose error and a pre-determined array of historical data points, to zero.

In certain aspects, monitoring a data stream including a set of contiguous source data points related to the analyte concentration includes monitoring a data stream received from a transcutaneously positioned analyte sensor, where the transcutaneously positioned analyte sensor in certain embodiments is operatively coupled to a continuous glucose monitoring system.

In certain embodiments, one or more sets of maximum smoothing signals are further utilized to determine acceleration data of the analyte concentration.

In accordance with another embodiment, there is provided a method for monitoring an analyte comprising monitoring a data stream including a set of contiguous source data points related to analyte concentration, providing one or more sets of maximum lag corrected signals from the set of contiguous source data points, wherein each set of maximum lag corrected signals is generated utilizing correction terms based on time derivative estimates and historical source data points, and wherein parameters for the maximum lag correction minimize the correlation between the expected glucose error and time derivative estimates and minimize the correlation between the expected glucose error and a pre-determined array of historical source data points, providing one or more sets of maximum smoothing signals from the set of contiguous source data points, wherein each set of maximum smoothing signals is generated utilizing a smoothing algorithm, determining analyte concentration utilizing the one or more sets of maximum lag corrected signals, and determining a rate of change in the analyte concentration utilizing the one or more sets of maximum smoothing signals.

In accordance with another embodiment of the present disclosure, there is provided a method for monitoring an analyte comprising monitoring a data stream including a set of contiguous source data points related to analyte concentration, providing one or more sets of maximum lag corrected signals from the set of contiguous source data points, wherein each set of maximum lag corrected signals is generated utilizing correction terms based on time derivative estimates and historical source data points, and wherein parameters for the maximum lag correction minimize the correlation between the expected glucose error and time derivative estimates and minimize the correlation between the expected glucose error and a pre-determined array of historical source data points, providing one or more sets of maximum smoothing signals from the set of contiguous source data points, wherein each set of maximum smoothing signals is generated utilizing a smoothing algorithm, determining analyte concentration utilizing a weighted combination of the one or more sets of maximum lag corrected signals and the one or more sets of maximum smoothing signals, wherein more weight is placed on the one or more sets of maximum lag corrected signals to determine the analyte concentration, and determining a rate of change in the analyte concentration utilizing a weighted combination of the one or more sets of maximum lag corrected signals and the one or more sets of maximum smoothing signals, where more weight is placed on the one or more sets of maximum smoothing signals to determine the rate of change.

In accordance with still another embodiment, there is provided a method for monitoring an analyte comprising monitoring a data stream including a set of contiguous source data points related to analyte concentration, providing one or more sets of maximum lag corrected signals from the set of contiguous source data points, wherein each set of maximum lag corrected signals is generated utilizing correction terms based on time derivative estimates and historical source data points, and wherein parameters for the maximum lag correction minimize the correlation between the expected glucose error and time derivative estimates and minimize the correlation between the expected glucose error and a pre-determined array of historical source data points, providing a first one or more sets of maximum smoothing signals from the set of contiguous source data points, wherein each set of maximum smoothing signals is generated utilizing a smoothing algorithm, providing a second one or more sets of maximum smoothing signals from the set of contiguous source data points, wherein each of the second set of maximum smoothing signals is generated utilizing a second smoothing algorithm, determining analyte concentration utilizing a weighted combination of the one or more sets of maximum lag corrected signals and the first and second one or more sets of maximum smoothing signals, wherein more weight is placed on the one or more sets of maximum lag corrected signals to determine the analyte concentration, and determining a rate of change in the analyte concentration utilizing a weighted combination of the one or more sets of maximum lag corrected signals and the first and second one or more sets of maximum smoothing signals, where more weight is placed on the first and second one or more sets of maximum smoothing signals to determine the rate of change.

In certain embodiments, the first smoothing algorithm and the second smoothing algorithm are different.

In certain embodiments, the method further includes averaging the first one or more sets of maximum smoothing signals and the second one or more sets of maximum smoothing signals.

An apparatus in accordance with another embodiment includes one or more processors, and a memory storing instructions which, when executed by the one or more processors, causes the one or more processors to monitor a data stream including a set of contiguous source data points related to the concentration of an analyte, to provide one or more sets of monitored data stream from the set of contiguous source data points, wherein each set of monitored data stream is generated utilizing a spline, up-sampling, or regularization algorithm, to provide one or more sets of maximum lag corrected signals from the one or more sets of monitored data stream, wherein each set of maximum lag corrected signals is generated utilizing correction terms based on time derivative estimates and historical monitored data stream data points, and wherein parameters for the maximum lag correction minimize the correlation between the expected glucose error and time derivative estimates and minimize the correlation between the expected glucose error and a pre-determined array of historical monitored data stream, to provide one or more sets of maximum smoothing signals from the set of monitored data stream, wherein each set of maximum smoothing signals is generated utilizing a smoothing algorithm, to determine analyte concentration utilizing the one or more sets of maximum lag corrected signals and to determine a rate of change in the analyte concentration utilizing the one or more sets of maximum smoothing signals.

In certain embodiments, the instructions to provide the one or more sets of maximum lag corrected signals include instructions to utilize an aggressive lag correction algorithm, the lag correction algorithm configured to drive one or more of the correlation between the expected glucose error and the time derivative estimates or the correlation between the expected glucose error and a pre-determined array of historical data points, to zero.

In certain embodiments, the instructions to monitor the data stream including the set of contiguous source data points related to the analyte concentration includes instructions to monitor a data stream received from a transcutaneously positioned analyte sensor operatively coupled to the one or more processors.

In certain embodiments, the one or more sets of maximum smoothing signals are further utilized to determine acceleration data of the analyte concentration.

An apparatus in accordance with another embodiment includes one or more processors, and a memory storing instructions which, when executed by the one or more processors, causes the one or more processors to monitor a data stream including a set of contiguous source data points related to analyte concentration, to provide one or more sets of maximum lag corrected signals from the set of contiguous source data points, wherein each set of maximum lag corrected signals is generated utilizing correction terms based on time derivative estimates and historical source data points, and wherein parameters for the maximum lag correction minimize the correlation between the expected glucose error and time derivative estimates and minimize the correlation between the expected glucose error and a pre-determined array of historical source data points, to provide one or more sets of maximum smoothing signals from the set of contiguous source data points, wherein each set of maximum smoothing signals is generated utilizing a smoothing algorithm, to determine analyte concentration utilizing the one or more sets of maximum lag corrected signals and to determine a rate of change in the analyte concentration utilizing the one or more sets of maximum smoothing signals.

An apparatus in accordance with still another embodiment includes one or more processors, and a memory storing instructions which, when executed by the one or more processors, causes the one or more processors to monitor a data stream including a set of contiguous source data points related to analyte concentration, to providing one or more sets of maximum lag corrected signals from the set of contiguous source data points, wherein each set of maximum lag corrected signals is generated utilizing correction terms based on time derivative estimates and historical source data points, and wherein parameters for the maximum lag correction minimize the correlation between the expected glucose error and time derivative estimates and minimize the correlation between the expected glucose error and a pre-determined array of historical source data points, to provide one or more sets of maximum smoothing signals from the set of contiguous source data points, wherein each set of maximum smoothing signals is generated utilizing a smoothing algorithm, to determine analyte concentration utilizing a weighted combination of the one or more sets of maximum lag corrected signals and the one or more sets of maximum smoothing signals, wherein more weight is placed on the one or more sets of maximum lag corrected signals to determine the analyte concentration and to determine a rate of change in the analyte concentration utilizing a weighted combination of the one or more sets of maximum lag corrected signals and the one or more sets of maximum smoothing signals, where more weight is placed on the one or more sets of maximum smoothing signals to determine the rate of change.

An apparatus in accordance with still yet another embodiment includes one or more processors, and a memory storing instructions which, when executed by the one or more processors, causes the one or more processors to monitor a data stream including a set of contiguous source data points related to analyte concentration, to provide one or more sets of maximum lag corrected signals from the set of contiguous source data points, wherein each set of maximum lag corrected signals is generated utilizing correction terms based on time derivative estimates and historical source data points, and wherein parameters for the maximum lag correction minimize the correlation between the expected glucose error and time derivative estimates and minimize the correlation between the expected glucose error and a pre-determined array of historical source data points, to provide a first one or more sets of maximum smoothing signals from the set of contiguous source data points, wherein each set of maximum smoothing signals is generated utilizing a smoothing algorithm, to provide a second one or more sets of maximum smoothing signals from the set of contiguous source data points, wherein each of the second set of maximum smoothing signals is generated utilizing a second smoothing algorithm, to determine analyte concentration utilizing a weighted combination of the one or more sets of maximum lag corrected signals and the first and second one or more sets of maximum smoothing signals, wherein more weight is placed on the one or more sets of maximum lag corrected signals to determine the analyte concentration and to determine a rate of change in the analyte concentration utilizing a weighted combination of the one or more sets of maximum lag corrected signals and the first and second one or more sets of maximum smoothing signals, where more weight is placed on the first and second one or more sets of maximum smoothing signals to determine the rate of change.

In certain embodiments, the first smoothing algorithm and the second smoothing algorithm are different.

Certain embodiment includes instructions to average the first one or more sets of maximum smoothing signals and the second one or more sets of maximum smoothing signals.

Various other algorithms and analysis may be applied in certain embodiments to the monitored analyte signal received from sensor 101 of analyte monitoring system 100 (FIG. 1) to correct for lag and noise. Further, the weight of lag corrected and smoothed signals are varied in embodiments in order to effectively maximize noise cancellation and system accuracy.

The present disclosure contemplates modifications as would occur to those skilled in the art. For example, it is contemplated that a variety of the lag correction algorithms and the smoothing algorithms disclosed herein can be developed, altered or otherwise changed utilizing the principles provided in the present disclosure, as would occur to those skilled in the art without departing from the spirit of the present disclosure. All publications, patents, and patent applications cited in this specification are herein incorporated by reference as if each individual publication, patent, or patent application was specifically and individually indicated to be incorporated by reference and set forth in its entirety herein.

Further, any theory of operation, proof, or finding stated herein is meant to further enhance understanding of the present disclosure and is not intended to make the scope of the present disclosure dependent upon such theory, proof, or finding. While the disclosure has been illustrated and described in detail in the figures and foregoing description, the same is considered to be illustrative and not restrictive in character, it being understood that only certain embodiments have been shown and described and that all changes and modifications that come within the spirit of the disclosure are desired to be protected.

What is claimed is:

1. A glucose monitoring system, comprising:
a glucose sensor configured to have at least a portion disposed in interstitial fluid of a user and generate data points related to a concentration of a glucose level in the interstitial fluid at a first sample rate;
a data processing device communicatively coupled with the glucose sensor; and
a receiver unit communicatively coupled with the data processing device, wherein the data processing device is configured to:
receive, from the glucose sensor, a first data stream comprising the data points related to the concentration of the glucose level in the interstitial fluid, wherein the first data stream is associated with the first sample rate;
wherein the data processing device or receiver unit are configured to:
generate a second data stream comprising data points related to the concentration of the glucose level in the interstitial fluid by processing the first data stream at a second sample rate, wherein the second sample rate is different than the first sample rate;
generate one or more first processed data points by applying one or more first data processing algorithms to the data points of the second data stream;
generate one or more second processed data points by applying one or more second data processing algorithms to the data points of the second data stream;
determine a value of the concentration of the glucose level based on a first weighted combination of one or more of the first processed data points and one or more of the second processed data points; and
determine a rate of change of the concentration of the glucose level based on a second weighted combination of one or more of the first processed data points and one or more of the second processed data points; and
wherein the receiver unit is configured to:
display the value of the concentration of the glucose level and rate of change of the concentration of the glucose level.

2. The system of claim 1, wherein the second sample rate is higher than the first sample rate, and wherein processing the first data stream comprises up-sampling the first data stream at the second sample rate.

3. The system of claim 1, wherein the one or more first data processing algorithms comprise a lag correction algorithm.

4. The system of claim 1, wherein the one or more second data processing algorithms comprise a data smoothing algorithm.

5. The system of claim 1, wherein the data processing device or receiver unit are further configured to provide the value of the concentration of the glucose level and rate of change of the concentration of the glucose level to a data processing terminal having a memory configured to receive the determined concentration of the glucose level, the data processing terminal comprising a drug delivery device configured to administer therapy based on the determined concentration of the glucose level.

6. The system of claim 1, wherein the receiver unit comprises a smartphone.

7. The system of claim 1, wherein the receiver unit comprises a wristwatch or armband.

8. The system of claim 1, wherein the first weighted combination and the second weighted combination include variable weights.

9. A method for monitoring a glucose level using a glucose monitoring system including a glucose sensor, a data processing device, and a receiver unit, the method comprising:
receiving, from the glucose sensor, a first data stream comprising data points related to a concentration of the glucose level in interstitial fluid of a user, wherein the first data stream is associated with a first sample rate;
generating a second data stream comprising data points related to the concentration of the glucose level in the interstitial fluid by processing the first data stream at a second sample rate, wherein the second sample rate is different than the first sample rate;
generating one or more first processed data points by applying one or more first data processing algorithms to the data points of the second data stream;
generating one or more second processed data points by applying one or more second data processing algorithms to the data points of the second data stream;
determining a value of the concentration of the glucose level based on a first weighted combination of one or more of the first processed data points and one or more of the second processed data points;
determining a rate of change of the concentration of the glucose level based on a second weighted combination of one or more of the first processed data points and one or more of the second processed data points; and
displaying, with the receiver unit, the value of the concentration of the glucose level and rate of change of the concentration of the glucose level.

10. The method of claim 9, wherein the glucose sensor is configured to have at least a portion disposed in the interstitial fluid of the user and to generate the data points related to the concentration of the glucose level in the interstitial fluid at the first sample rate; and
wherein the data processing device is communicatively coupled with the glucose sensor and the receiver unit.

11. The method of claim 9, wherein the second sample rate is higher than the first sample rate, and wherein processing the first data stream comprises up-sampling the first data stream at the second sample rate.

12. The method of claim 9, wherein the one or more first data processing algorithms comprise a lag correction algorithm.

13. The method of claim 9, wherein the one or more second data processing algorithms comprise a data smoothing algorithm.

14. The method of claim 9, further comprising:
providing the value of the concentration of the glucose level and rate of change of the concentration of the glucose level to a data processing terminal having a memory configured to receive the determined concentration of the glucose level and comprising a drug delivery device; and
administering, using the data processing terminal and drug delivery device, therapy based on the determined concentration of the glucose level.

15. The method of claim 9, wherein the receiver unit comprises a smartphone.

16. The method of claim 9, wherein the receiver unit comprises a wristwatch or armband.
